(19) United States
(12) Patent Application Publication
(10) Pub. No.: US 2022/0346764 A1
(43) Pub. Date: Nov. 3, 2022

(54) TISSUE RETRACTOR DEVICE

(71) Applicant: Nextremity Solutions, Inc., Warsaw, IN (US)

(72) Inventor: Thomas HOLTON, Winona Lake, IN (US)

(73) Assignee: Nextremity Solutions, Inc., Warsaw, IN (US)

(21) Appl. No.: 17/659,931

(22) Filed: Apr. 20, 2022

Related U.S. Application Data
(60) Provisional application No. 63/183,232, filed on May 3, 2021.

(51) Int. Cl. A61B 17/02; A61B 17/08

(52) U.S. Cl. CPC A61B 17/02; A61B 17/08; A61B 2017/0287

(57) ABSTRACT

A tissue retractor device includes a first retractor, a second retractor and a band extending between the first retractor and the second retractor. The first and second retractors are configured to grasp edges of an incision while the band is configured to form an anatomy space and wrap around a patient's body part, while. At least one of the first retractor and the second retractor includes a first arcuate portion, a first connection portion and a first cam portion positioned between the first arcuate portion and the first connection portion. The first cam portion includes a cam surface for contacting the skin of a patient outside of an incision. The length of the band extending between the first retractor and the second retractor is adjustable.

### CROSS REFERENCE TO RELATED APPLICATIONS

The present application claims priority to U.S. Provisional Application Ser. No. 63/183,232 filed on May 3, 2021 (Attorney Docket No. 3768.107P1), which is incorporated herein by reference in their entireties

### BACKGROUND OF THE INVENTION

### Technical Field

The present invention generally relates to a tissue retractor device and, more particularly, to a self-retained, circumferential tissue retractor device used for maintaining any incision in an open position during a surgical procedure.

### Background Information

Retraction of tissues and anatomical structures is a critical part of all forms of surgery. Retraction requires adequate traction and counter-traction of tissues to expose, for example, the tissue or organ under examination for a surgeon.

Surgical retractors assist surgeons and operating room professionals hold an incision or wound open during surgical procedures. Surgical retractors aid in holding back and exposing underlying organs or tissues, allowing better visibility and access to the exposed area.

Currently, there are two types of surgical retractors that are commonly used, namely, hand-held retractors and self-retaining retractors. Hand-held retractors must be held manually by an assistant, robot, or surgeon during a surgical procedure. These types of retractors are cumbersome and limited in retention and retractor mobility for soft tissue. Also, hand-held retractors are large, heavy and obstruct imaging under fluoroscopy.

Self-retaining retractors attempt to keep an incision open by staying open on their own without assistance from an assistant, robot, or surgeon. Typically, self-retaining retractors require a screw or some type of clamp to hold the tissue by itself that provides a surgeon with two free hands. Examples of some self-retaining retractors used today are Weitlaner retractors, Cerebellar retractors, Beckmann retractors and Willams retractors. However, current self-retaining retractors are rigid and only provide retraction in one or two planes. Also, current self-retaining retractors obstruct fluoroscopy imaging.

Thus, a need exists for a tissue retractor to be used for surgical procedures that provides, for example, variable self-retaining length and/or tension with the ability to flex and move in multiple planes with a patient's tissue. There is also a need for a tissue retractor that provides less obstruction imagining under fluoroscopy.

### SUMMARY OF THE INVENTION

Briefly, the retractor device constructed in accordance with one or more aspects of the present invention satisfies the need for applying variable self-retaining tension to a patient's tissue with the ability to flex and move in multiple planes with a patient's tissue during a surgical procedure. The retractor device constructed in accordance with one or more aspects of the present invention also satisfies the need for providing less obstruction imagining under fluoroscopy.

In one embodiment, a tissue retractor device includes a first retractor, a second retractor and a band. The first retractor includes a first arcuate portion, a first connection portion and a first cam portion. The first cam portion is positioned between the first arcuate portion and the first connection portion. The first cam portion includes a first cam surface. The first arcuate portion is configured to grasp a first edge of an incision. The second retractor is configured to grasp a second edge of an incision. The band is configured to wrap around a patient's body part. The band extends between the first connection portion of the first retractor and the second retractor to form an anatomy space. The patient's body part is received within the anatomy space during a surgery. At least one of the first retractor and the second retractor includes a through hole. The through hole including a first portion sized to allow passage of the band and a second portion sized to retain the band.

In one example, the through hole includes a tapered slot extending from the first portion of the through hole to the second portion of the through hole.

In another example, the first cam surface is configured to pivot on skin outside of an incision based on tension created by the band. In yet another example, the cam surface is U-shaped.

In another example, the first arcuate portion of said first retractor includes a plurality of finger hooks, the plurality of finger hooks configured to grasp an edge of incised skin.

In another example, the tissue retractor device may have multiple retractors grasping the same edge of an incision.

These, and other objects, features and advantages of this invention will become apparent from the following detailed description of the various aspects of the invention taken in conjunction with the accompanying drawings.

### BRIEF DESCRIPTION OF THE DRAWINGS

The present invention will be understood more fully from the detailed description given hereinafter and from the accompanying drawings of the certain embodiments of the present invention, which, however, should not be taken to limit the invention, but are for explanation, illustration and understanding only.

FIG. 1 depicts a perspective view of one embodiment of a tissue retractor device constructed in accordance with one or more aspects of the present invention;

FIG. 2 depicts a side view of the tissue retractor device shown in FIG. 1;

FIG. 2A depicts a side view of an alternative tissue retractor device constructed in accordance with one or more aspects of the present invention;

FIG. 3 depicts a perspective view of another embodiment of a tissue retractor device constructed in accordance with one or more aspects of the present invention;

FIG. 4A depicts a perspective view of one embodiment of a tissue retractor constructed in accordance with one or more aspects of the present invention;

FIG. 4B depicts a side view of the tissue retractor constructed in accordance with one or more aspects of the present invention;

FIG. 5A depicts perspective view of an alternative example of a through hole for retaining a band for a tissue retractor device constructed in accordance with one or more aspects of the present invention;

FIG. 5B depicts a side view of the example illustrated in FIG. 5A;

FIG. 6 depicts a perspective view of another embodiment of a tissue retractor constructed in accordance with one or more aspects of the present invention;

FIG. 7 depicts a side view of one embodiment of an adjustment member constructed in accordance with one or more aspects of the present invention;

FIG. 8A depicts a perspective view of an alternative embodiment of an adjustment member constructed in accordance with one or more aspects of the present invention;

FIG. 8B depicts a side view of the adjustment member illustrated in FIG. 8A with a looped portion of one example of a band extending therefrom;

FIG. 8C depicts a side view of the adjustment member illustrated in FIG. 8A with two bands extending therefrom;

9 depicts a perspective view of one example of a tissue retractor device constructed in accordance with one or more aspects of the present invention in use with an open incision of a patient's foot; and

FIG. 10 depicts a cross-sectional view of the tissue retractor device in use taken along the plane 9-9 shown in FIG. 9.

### DETAILED DESCRIPTION OF THE INVENTION

The present invention will be discussed hereinafter in detail in terms of various exemplary embodiments according to the present invention with reference to the accompanying drawings. In the following detailed description, numerous specific details are set forth to provide a thorough understanding of the present invention. It will be obvious, however, to those skilled in the art that the present invention may be practiced without some of these specific details. In other instances, well-known structures are not shown in detail to avoid unnecessary obscuring of the present invention.

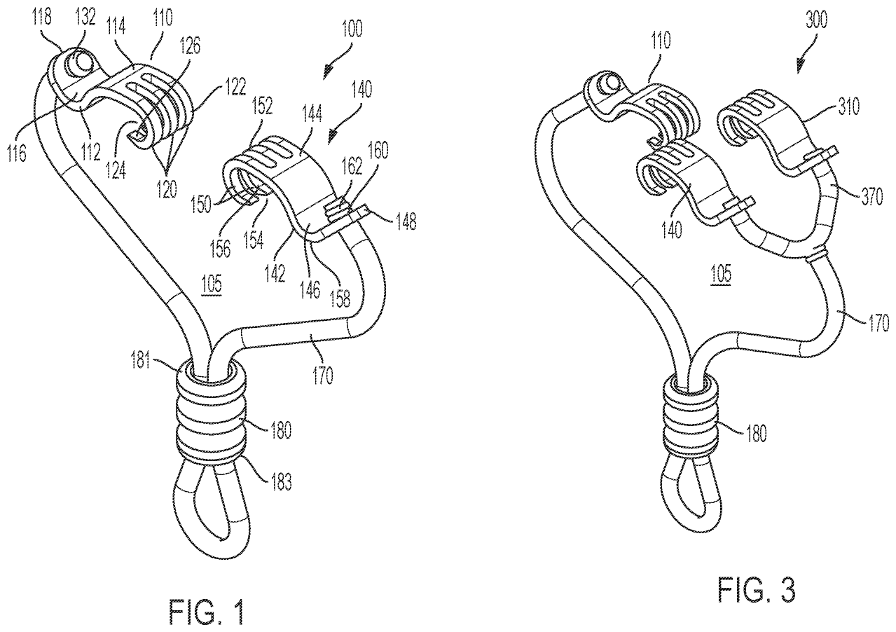

Thus, all implementations described below are exemplary implementations provided to enable persons skilled in the art to make or use the embodiments of the disclosure and are not intended to limit the scope of the disclosure, which is defined by the claims. As used herein, the word “exemplary” or “illustrative” or “example”, and derivatives thereof, means “serving as an example, instance, or illustration.” Any implementation described herein as “exemplary” or “illustrative” or “example”, and derivatives thereof, is not necessarily and should not be construed as preferred or advantageous over other implementations. Moreover, in the present description, the terms “upper”, “lower”, “left”, “rear”, “right”, “front”, “vertical”, “horizontal”, and derivatives thereof shall relate to the invention as oriented in FIG. 1.

Furthermore, there is no intention to be bound by any expressed or implied theory presented in the preceding technical field, background, brief summary or the following detailed description. It is also understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise. While this invention is satisfied by embodiments in many different forms, there is shown in the drawings, and will herein be described in detail, one or more embodiments of the present invention with the understanding that the present disclosure is to be considered as exemplary of the principles and aspects of the invention and is not intended to limit the invention to the embodiments illustrated. The scope of the invention will be pointed out in the appended claims.

In short, a tissue retractor device constructed in accordance with one or more aspects of the present invention provides, for example, variable, self-retaining tension to a patient's tissue with the ability to flex and move in multiple planes with a patient's tissue during a surgical procedure. A tissue retractor device constructed in accordance with one or more aspects of the present invention also satisfies the need for providing less obstruction imagining under fluoroscopy.

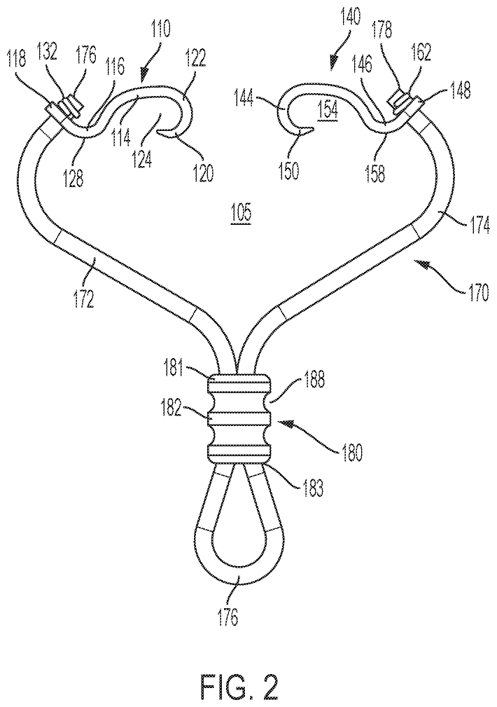

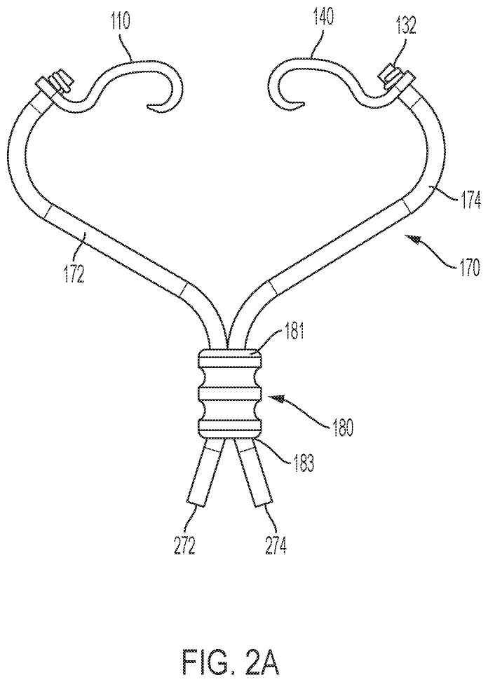

Referring now to FIGS. 1 and 2, there is shown one example of a tissue retractor device 100 constructed in accordance with one or more aspects of the present invention. Tissue retractor device 100 is suitable for use during surgery against a body member, including, for example, a foot, a leg, an arm, a hand, a torso and/or a head.

As illustrated in FIGS. 1 and 2, tissue retractor device 100 includes a first retractor 110, a second retractor 140 and a band 170 extending between first retractor 110 and second retractor 140. Tissue retractor device 100 may also include an adjustment member 180 slidably receiving and retaining portions of band 170 to move closer to or away from first retractor 110 and/or second retractor 140, as will be described in more detail below. In alternative embodiments, as discussed in more detail below, the length and tension of band 170 between first and second retractors 110, 140 may be adjusted where at least one end of band 170 slidably couples to at least one of first and second retractors 110, 140.

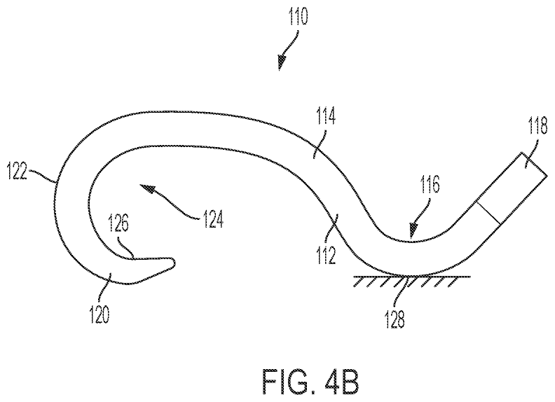

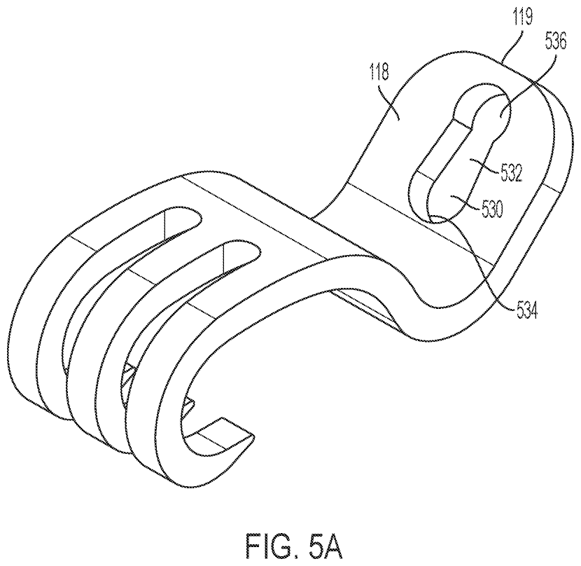

One example of a first retractor 110 is illustrated in FIGS. 4A and 4B. In this example, first retractor 110 includes a body 112 comprising an arcuate section 114, a cam section 116, and a connection section 118. Arcuate section 114 extends upwardly and outwardly from cam portion 116 towards a plurality of finger hooks or prongs 120. Finger hooks or prongs 120 extend through a bend 122 and are arranged in spaced parallel relationship to each other to form, for example, a soft tissue space 124. Together, finger hooks or prongs 120 may form a rack-like structure. The distribution of fingers or prongs 120 in a spaced parallel relationship effectively grasp tissue within soft tissue space 124 while minimizing damage to the tissue contained therein. In one example, each finger or prong 120 may include a grasping surface 126 including one or more ribs (not shown) extending outwardly from the interior surface to assist in grasping and/or retaining tissue. The ends of each finger or prong 120 may also taper to form a wedge shaped for assisting to grasp or hold tissue.

Cam section 116 of first retractor 110 may include a U-shaped configuration having a cam surface 128 for, during use, pivotably contacting skin at a proximal distance from an incision in response to tension provided by band 170. A U-shaped cam surface 128 provides smooth contact with the skin and avoids puncturing the skin. Cam surface 128 provides leverage to arcuate portion 114 of first retractor 110 in response to the tension applied to band 170 by a surgeon.

Connection section 118 extends upwardly and outwardly from cam section 116. A through hole 130 may be formed in connection section 116 of body 112. Hole 130 may be centered in connection section 118 and includes a diameter large enough to receive band 170. In one example, end 176 of band 170 is received by hole 130 and retained in place by a retaining clasp 132 that prevents band 170 from falling out of hole 130 during use.

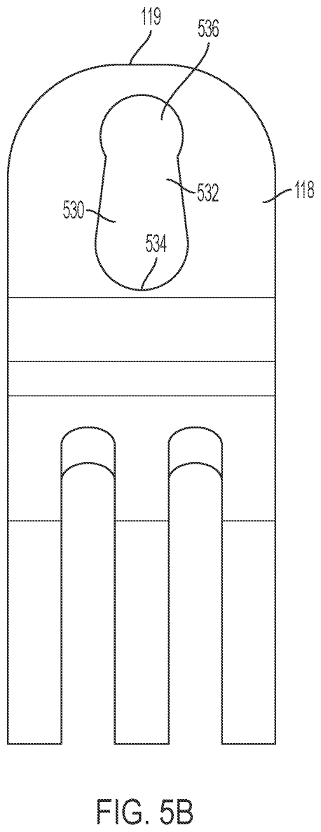

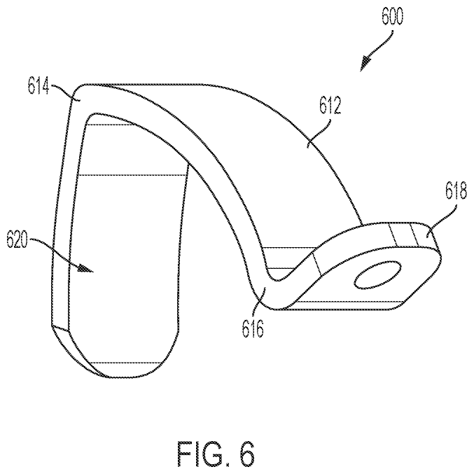

In an alternative embodiment illustrated in FIGS. 5A and 5B, through hole 530 may have a diameter somewhat larger than the diameter of band 170. In this example, a slot 532 may open to hole 530 and extend downwardly towards an outer edge 119 of connection section 118. Slot 532 may be tapered having a wide end opening 534 to the interior of hole 530 and a narrow end 536 adjacent to outer edge 119, forming a key-hole shaped opening. Narrow end 536 may include a diameter smaller than the diameter of band 170 to resiliently retain or hold band 170 and prevent band 170 from moving. Slot 532 may be configured so that when a downward pushing force is applied to an end 172 of band 170, end 172 of band 170 will be compressively or resiliently fixed in position in narrow end 536. In this embodiment, a separate adjustment member is not necessary or required. In contrast, band 170 extends between first retractor 110 to second retractor 140. The tension of band 170 around, and anatomy space for receiving, a patient's limb is adjusted by pulling end 172 of band 170 further through hole 530 and fixing it in place within narrow end 536 through slot 532.

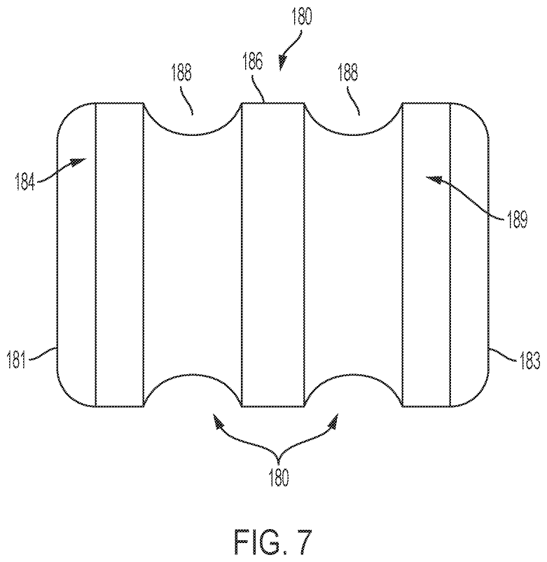

FIG. 6 depicts another example of a first retractor 600 constructed in accordance with one or more aspects of the present invention. In this example, first retractor 600 includes a body 612 comprising an arcuate section 614, a cam section 616, and a connection section 618. Arcuate section 614 extends upwardly and outwardly from cam portion 616 towards a smooth soft tissue paddle 620. Cam section 616 and connection section 618 may be constructed or have a similar configuration as cam section 116 and connection section 118 of first retractor 110.

Second retractor 140 may be similarly constructed or have a similar configuration as first retractors 110 and/or 600. For example, second retractor 140 includes a body 142 comprising an arcuate section 144, a cam section 146, and a connection section 148. Arcuate section 144 extends upwardly and outwardly from cam portion 146 towards a plurality of finger hooks or prongs 150. Finger hooks or prongs 150 extend through a bend 152 and are arranged in spaced parallel relationship to each other to form, for example, a soft tissue space 154. The distribution of fingers or prongs 150 in a spaced parallel relationship effectively grasp tissue within soft tissue space 154 while minimizing damage to the tissue contained therein. In one example, each finger or prong 150 may include a grasping surface 156 including one or more ribs (not shown) extending outwardly from the interior surface to assist in grasping and/or retaining tissue.

Cam section 146 of second retractor 140 may include a U-shaped configuration having a cam surface 158 for, during use, pivotably contacting skin at a proximal distance from an incision. Connection section 148 extends upwardly and outwardly from cam section 146. A through hole 160 may be formed in connection section 146 of body 142. Hole 160 may be centered in connection section 148 and include a diameter large enough to receive band 170. In one example, end 178 of band 170 is received by hole 160 and retained in place by a retaining clasp 162 that prevents band 170 from falling out of hole 160 during use. In an alternative embodiment, through hole may be similarly constructed and configured as through hole 530, slot 532 and narrow end 536 illustrated in FIGS. 5A and 5B. On this side, the tension of band 170 around a patient's limb is adjusted by pulling end 178 of band 170 further through hole 530 and fixing it in place within narrow end 536 through slot 532. In yet another embodiment, only one of first retractor 110 and second retractor 140 includes such a through hole, while the other includes a typical through hole that retains an end of band 170 by use of a retaining clasp.

As illustrated in FIGS. 1 and 2, first retractor 110 and second retractor 140 are joined by band 170 to form an anatomy space 105. Band 170 may be an elastic or non-elastic strip that, in use, is wrapped around the anatomy, e.g. foot, of a patent, with the specific anatomy positioned within anatomy space 105. In one example, band 170 may be surgical tubing, such as, for example, a non-latex rubber tubing. Each end 176, 178 of band 170 is pulled through holes 130, 160 formed in connection sections 116, 146 of first retractor 110 and second retractor 140, respectively, and held by clasps 132, 162.

In one example illustrated in FIGS. 1 and 2, band 170 may include a first retractor portion 172 extending from first retractor 110, a second retractor portion 174 extending from second retractor 140, and a loop portion 176 connecting first retractor portion 172 and second retractor portion 174. In this example, tissue retractor device 100 includes an adjustment member 180. Adjustment member 180 may be used to increase or decrease anatomy space 105 formed by tissue retractor device 100 by sliding adjustment member 180 along looped portion 176 of band 170.

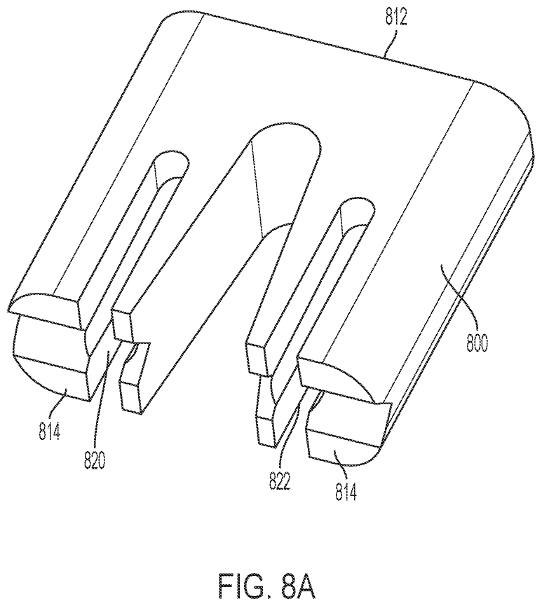

In one embodiment as illustrated in FIGS. 1 and 7, adjustment member 180 may include a cylindrical body 182 having an opening 184 extending between a first side 181 and a second side 183 through which extends a portion of loop portion 176 of band 170. With a portion of loop portion 176 of band 170 extending through opening 184 and out second side 183, adjustment member 180 may be slid along band 170 to be either in closer proximity or farther away from first retractor 110 and/or second retractor 140. Cylindrical body 182 of adjustment member 180 includes an outer surface 186 having one or more knurling 188 to provide a better grip of adjustment member 180 for a surgeon's hand or fingers.

When adjustment member 180 is slidably moved closer to first retractor 110 and/or second retractor 140, anatomy space 105 reduces in size and loop portion 176 extending out of second side 183 of adjustment member 180 increases, causing tissue retractor device 100 to tighten around the desired anatomy and apply more tension to first and second retractors 110, 140. When adjustment member 180 is slidably moved away from first retractor 110 and/or second retractor 140, anatomy space 105 is increased and loop portion 176 extending out of second side 183 of adjustment member 180 decreases, causing tissue retractor device 100 to loosen from the desired anatomy and apply less tension.

In an alternative embodiment illustrated in FIG. 2A, first retractor portion 172 of band 170 extends from first retractor 110 and passes through opening 184 of adjustment member 180 and out second side 183 of adjustment member 180, and second retractor portion 174 extends from second retractor 110 and passes through opening 184 of adjustment member 180 and out second side 183 of adjustment member 180. In this embodiment, first retractor portion 172 and second retractor portion 174 of band 170 do not connect. Instead, first retractor portion 172 includes a free end 272 and second retractor portion 174 includes a free end 274. Free ends 272, 274 extend beyond second side 183 of adjustment member 180 for gripping by a surgeon, together or independently, while sliding adjustment member 180 along portions of first and section retractor portions 172, 174 of band 170.

In one embodiment, through hole(s) 184 of adjustment member 180 may be tapered and/or include an interior friction surface 189 to resiliently retain band 170 in a desired location when not being manually slid along band 170. By using an adjustment member 180 constructed in accordance with one or more aspects of the present invention, the tension applied to the retracted incision by first retractor 110 and second retractor 140 is easily controlled by sliding adjustment member 180 along band 170. In use, adjustment member 180 would be located on the opposite side of the anatomy from any incision to avoid any disruption to the incision site and/or engagement of first and second retractors 110, 140 or the tissue at the incision site.

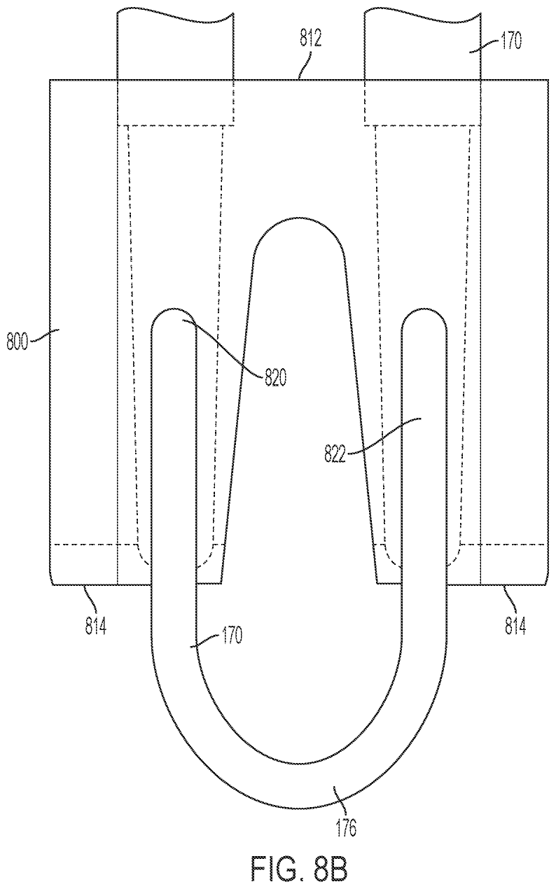

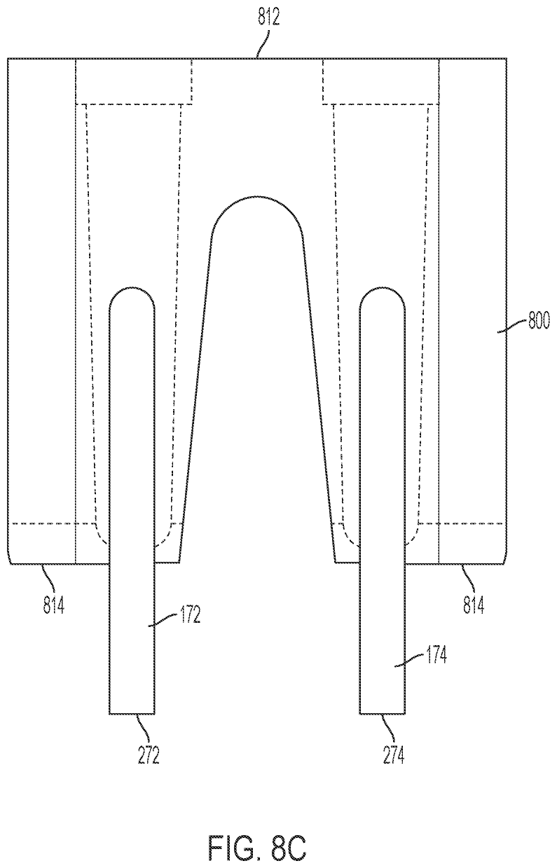

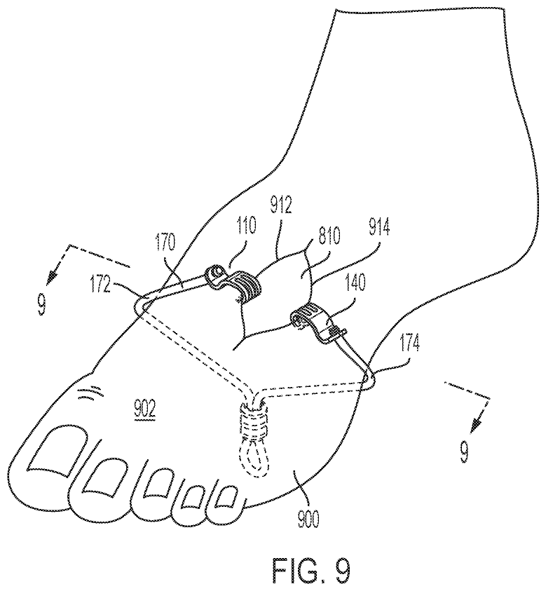

An alternative example of an adjustment member 800 is illustrated in FIGS. 8A and 8B. In this embodiment, adjustment member 800 may include a body 810 having a first opening 820 and a second opening 822 extending between a first side 812 and a second side 814 through which extends portions of band 170. With a portion of band 170 extending through first opening 820 and another portion of band 170 extending through second opening (see e.g. FIG. 8B), adjustment member 800 may be slid along band 170 to be either in closer proximity or farther away from first retractor 110 and/or second retractor 140. First and second openings 820, 822 may be tapered and/or include an interior friction surface to resiliently retain band 170 in a desired location when not being manually slid along band 170

When adjustment member 800 is slidably moved closer to first retractor 110 and/or second retractor 140, anatomy space 105 reduces in size and loop portion 176 extending out of second side 814 of adjustment member 800 increases, causing tissue retractor device 100 to tighten around the desired anatomy and apply more tension to first and second retractors 110, 140. When adjustment member 800 is slidably moved away from first retractor 110 and/or second retractor 140, anatomy space 105 is increased and loop portion 176 extending out of second side 814 of adjustment member 800 decreases, causing tissue retractor device 100 to loosen from the desired anatomy and apply less tension.

Alternatively, first retractor portion 172 of band 170 extends from first retractor 110 and passes through first opening 820 of adjustment member 800 and out second side 814 of adjustment member 800, and second retractor portion 174 extends from second retractor 110 passes through second opening 822 of adjustment member 800 and out second side 814 of adjustment member 800. In this embodiment, first retractor portion 172 and second retractor portion 174 of band 170 do not connect. Instead, first retractor portion 172 includes a free end 272 and second retractor portion 174 includes a free end 274. Free ends 272, 274 extend beyond second side 814 of adjustment member 800 for gripping by a surgeon, together or independently, while sliding adjustment member 800 along portions of first and section retractor portions 172, 174 of band 170.

Adjustment members 180 and 800 are only examples of structures that could be used to adjust the size of anatomy space 105 and the tension of band 170 between first retractor 110 and second retractor 140. Alternative designs including one or more through holes, including pinch, roller, slide, screw or flange clamps, clips or ties, for receiving portions of band 170 may be used to accomplish the same purpose of adjusting the size of anatomy space 105 and the tension of band 170 between first retractor 110 and second retractor 140

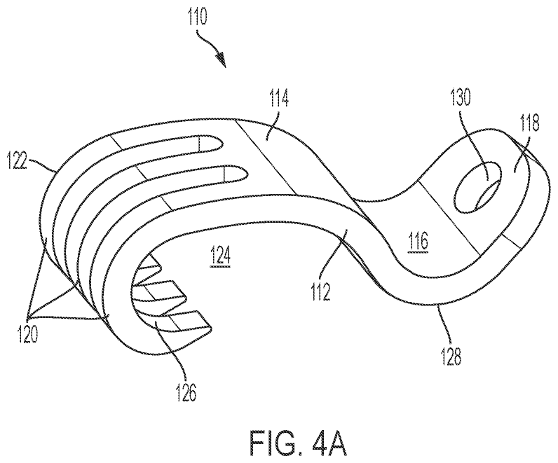

FIG. 3 illustrates another example of a tissue retractor device 300. In this example, a third retractor 310 may be used to assist in retracting additional portions of a larger incision. Third retractor 310 is connected to band 170 by a second band 370 extending from a connection section 318 of third retractor 310 and joined and/or otherwise connected to band 170. In another example, second band 370 may be formed integrally with band 170. In alternative embodiments, additional retractors (including the retractors illustrated in FIGS. 4A and 6 or other retractor shapes and configurations) may be provided or used on either side of tissue retractor device 100 and connected to band 170 depending on need and/or size of the incision.

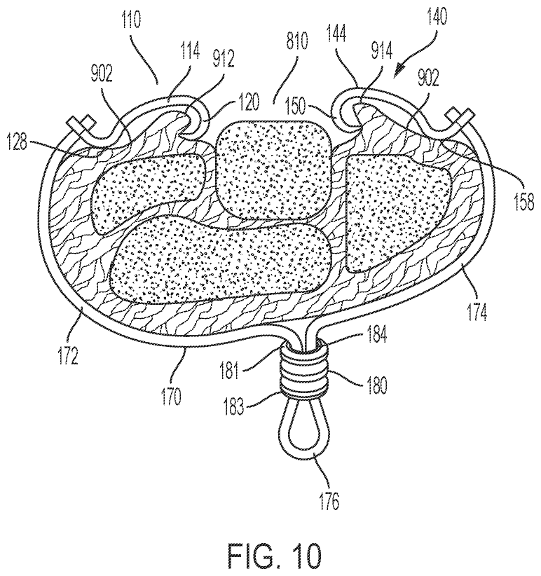

FIGS. 9 and 10 show the use of a tissue retractor device 100 in association with a surgical procedure. In FIGS. 9 and 10, a dorsal incision 910 is formed through skin 902 on top of a patient's foot 900. When this incision 910 is made, the skin will have a first edge 912 and a second edge 914 opposite to and separated from first edge 912. After the incision 910 is made, first retractor 110 can be placed over first edge 912 such that finger hooks or prongs 120 of first retractor 110 will extend to be positioned adjacent to the interior surface of skin 902 and first edge 912 of the incision 910 is positioned within soft tissue space 124 formed by arcuate portion 114. Arcuate portion 114 extends upwardly across and rearwardly from first edge 912 of the incised skin away from incision 910, and then downwardly towards cam portion 116. As can be seen in FIG. 10, cam portion 116 of first retractor 110 contacts the outer surface of skin 902 at a distance outside first edge 912 of incision 910.

Band 170, held to connection portion 118 of first retractor 110 by clasp 132, passes through hole 130 and extends circumferentially around the plantar portion of a patient's foot to second retractor 140. As illustrated, first retractor portion 172 of band 170 extends from first retractor 110 to adjustment member 180, second retractor portion 174 of band 170 extends from second retractor 140 to adjustment member 180, and loop portion 176 of band 170 extends through opening 184 and out of second side 185 of adjustment member 180. At least a portion of loop portion 176 of band 170 extends and remains out of second side 183 of adjustment member 180 to enable a surgeon to grab and adjust tension on first and second retractors 110, 140, as discussed in more detail below. End 178 of second retractor portion 174 of band 170 passes through hole in connection portion 148 and is held in place by clasp 162.

In an alternative embodiment illustrated in FIG. 2A, both first retractor portion 172 and second retractor portion 174 extend from first and second retractors 110, 140 respectively, and pass from first side 181 of adjustment member 180 through opening 184 and out second side 185 of adjustment member 180. The ends 272, 274 of first retractor portion 172 and second retractor portion 174, respectively extend and remain out of second side 185 of adjustment member 180 to enable a surgeon to grab and adjust tension on first and second retractors 110, 140.

Second retractor 140 grasps second edge 914 of incision in a similar manner as did the first retractor 110 to first edge 912. As illustrated in FIGS. 9 and 10, cam portion 146 of second retractor 140 contacts the outer surface of skin 902 at a distance outside second edge 912 of incision 910. The number of retractors and/or the number of hooks or prongs, or in alternative embodiment, paddles or other retractor grasping configurations, of each retractor may vary for each side of the incision depending on the size of the incision.

After first retractor 110 grasps first edge 912 of incision 910 and second retractor 140 grasp second edge 914 of incision 910, a surgeon can adjust the length of band 170 around foot 900 to the desired degree necessary to create the proper tension for pulling first and second edges 912, 914 of skin 902 apart by, with one hand or finger, grabbing loop portion 176 and, with the other hand, sliding adjustment member 180, with enough force to overcome the friction of interior surface 189, towards the bottom of patient's foot 900 to decrease anatomy space 105. As adjustment member 180 is slid along loop portion 176 of band 170 by the surgeon, cam portions 116, 146 of first and second retractors 110, 140, respectively, pivot on the outer skin 902 away from incision 910 to provide mechanical leverage to apply a force to each retractor 110, 140. Once the desired degree of tension is achieved, band 170 will be maintained in position within adjustment member 180 by, for example, friction by interior surface 189, around foot 900.

The surgeon can then carry out the necessary surgical procedure within incision 910 or take clear imaging fluoroscopy without obstruction for tissue retraction device 100. After surgery is completed, the surgeon can simply slide adjustment member 180 away from the bottom of foot 900 to alleviate the tension in band 170 and increase anatomy space 105 and allow first retractor 110 and second retractor 140 to be removed or pulled from first and second edges 912, 914, respectively, of incision 910. Incision 910 can then be closed in a conventional surgical manner by, for example, stiches or staples.

In yet another embodiment (not shown), a first retractor constructed in accordance with one or more aspects of the present invention may include an arcuate section that attaches to tissue to be retracted, as described above, while its connection portion couples to a band that extends between and couples to a fixed object, e.g. an operating table, in the operating room. In this example, one end of the band may be coupled to the first retractor by a clasp 132 or by a through hole similar to the key-shaped hole illustrated in FIGS. 5A and 5B, while the other end of the band may tie to the fixed object, or couple to a clamp, clip or other attachment mechanism, by tying, a clasp or a through hole similar to the key-shaped hole illustrated in FIGS. 5A and 5B, attached to the fixed object. Adjustment of the tension on the band may be altered or changed by adjusting the length of the band extending between the fixed object and first retractor. This may be accomplished by using, for example, a through hole similar to the key-shaped hole illustrated in FIGS. 5A and 5B on one or both of the first retractor and attachment mechanism coupled to the fixed object or, alternatively, changing where the attachment mechanism couples to the fixed object (e.g. either closer or farther away from the first retractor) to vary the length, and tension, of the band extending therebetween.

A tissue retractor device constructed in accordance with one or more aspects of the present invention achieves several advantages over conventional manual or self-retaining retractors. For example, since there is no structure above an incision to create an obstacle to the surgical procedure, the incision will be virtually free of obstacles and clear for imaging fluoroscopy. The unique shape and configuration of first retractor and second retractor, especially cam sections, minimize damage to tissue adjacent the incision.

While several aspects of the present invention have been described and depicted herein, alternative aspects may be affected by those skilled in the art to accomplish the same objectives. Accordingly, it is intended by the appended claims to cover all such alternative aspects as fall within the true spirit and scope of the invention.

What is claimed:

1. A tissue retractor device, said tissue retractor device comprising:
a first retractor, said first retractor including a first arcuate portion, a first connection portion and a first cam portion, the first cam portion positioned between the first arcuate portion and the first connection portion, the first cam portion including a first cam surface configurated to contact skin outside of an incision during a surgery, the first arcuate portion configured to grasp a first edge of an incision;
a second retractor, said second retractor configured to grasp a second edge of an incision;
a band, said band configured to wrap around a patient's body part, said band extending between the first connection portion of said first retractor and said second retractor to form an anatomy space, wherein the patient's body part is received within the anatomy space during a surgery, wherein the first connection portion of said first retractor includes a through hole, the through hole including a first portion sized to allow passage of said band to adjust a size of the anatomy space and a second portion sized to retain said band.

2. The tissue retractor device of claim 1, wherein the through hole includes a tapered slot extending from the first portion of the through hole to the second portion of the through hole.

3. The tissue retractor device of claim 1, wherein said band is retained by said second retractor by a clasp.

4. The tissue retractor device of claim 1, wherein the first cam surface is configured to pivot on skin outside of an incision based on tension created by said band.

5. The tissue retractor device of claim 1, wherein the first cam surface is U-shaped.

6. The tissue retractor device of claim 1, wherein the first arcuate portion of said first retractor includes a plurality of finger hooks, the plurality of finger hooks configured to grasp an edge of incised skin.

7. A tissue retractor device, said tissue retractor device comprising:
a first retractor, said first retractor including a first arcuate portion, a first connection portion and a first cam portion, the first cam portion positioned between the first arcuate portion and the first connection portion, the first cam portion including a first cam surface;
a second retractor;
a band, said band extending between the first connection portion of said first retractor and said second retractor; and
an adjustment member, said adjustment member including a body, the body defining at least one through hole for receiving a portion of said band, said adjustment member slidable along a looped portion of said band towards and away from at least one of said first retractor or said second retractor to vary a distance between said first retractor and said second retractor.

8. The tissue retractor device of claim 7, wherein the first cam surface is configured to pivot on skin outside of an incision based on tension created by slidably movement of said adjustment member along said band.

9. The tissue retractor device of claim 7, wherein the first cam surface is U-shaped.

10. The tissue retractor device of claim 7, wherein the first arcuate portion of said first retractor includes a plurality of finger hooks, the plurality of finger hooks configured to grasp an edge of incised skin.

11. The tissue retractor device of claim 7, wherein the through hole of said adjustment member includes a friction interior surface.

12. The tissue retractor device of claim 7, wherein the through hole of said adjustment member is tapered.

13. The tissue retractor device of claim 7, further comprising a third retractor connected to a second band, the second band connected to said band.

14. The tissue retractor device of claim 7, wherein the first arcuate portion of said first retractor includes a soft tissue paddle, the soft tissue paddle configured to grasp an edge of incised skin.

15. A self-retained circumferential tissue retractor device, said tissue retractor device comprising:
a first retractor, said first retractor including a first arcuate portion, a first connection portion and a first cam portion, the first cam portion positioned between the first arcuate portion and the first connection portion, the first cam portion including a first cam surface;
a second retractor;
a first band, said first band including a first end and a second end, the first end of said first band coupled to said first retractor, said first band extending from said first retractor;
a second band, said second band including a first end and a second end, the first end of said second band coupled to said second retractor, said second band extending from said second retractor;
an adjustment member, said adjustment member including a body, the body including a first side and a second side, the body defining a through hole, a portion of said first band passing from the first side through the through hole of the body of said adjustment member, a portion of said second band passing from the first side through the through hole of the body of said adjustment member, wherein the second end of said first band and the second end of said second band extend out of the second end of said adjustment member.

16. The self-retained circumferential tissue retractor device of claim 15, wherein the second end of said first band connects to the second end of said second band to create a loop section.
